(19) United States
(12) Patent Application Publication
(10) Pub. No.: US 2024/0157123 A1
(43) Pub. Date: May 16, 2024

(54) WEARABLE INSOLE FOR ENHANCED DIABETIC FOOT ULCER WOUND HEALING

(71) Applicant: Board of Trustees of Western Michigan University, Kalamazoo, MI (US)

(72) Inventors: Massood Z. Atashbar, Portage, MI (US); Dinesh Maddipalta, Portage, MI (US); Binu Baby Narakathu, Portage, MI (US); Simin Masihi, Kalamazoo, MI (US); Masoud Panahi, Kalamazoo, MI (US); Daryl Lawson, Kalamazoo, MI (US); Jose Rodriguez-Labra, Portage, MI (US); Anthony Hanson, Belmont, MI (US)

(21) Appl. No.: 18/509,207

(22) Filed: Nov. 14, 2023

Related U.S. Application Data
(60) Provisional application No. 63/425,073, filed on Nov. 14, 2022.

(51) Int. Cl. A61N 1/04; A43B 3/44; A43B 7/00; A43B 17/02; A43B 17/14; A43D 8/02; A61F 7/00; A61N 1/36; H01B 5/14

(52) U.S. Cl. CPC A61N 1/0484; A43B 3/44; A43B 7/00; A43B 17/02; A43B 17/14; A43D 8/02; A61F 7/007; A61N 1/0468; A61N 1/0476; A61N 1/36031; A61N 1/36034; H01B 5/14; A61F 2007/0045; A61F 2007/0233

(57) ABSTRACT

A shoe insole includes photoplethysmography (PPG) sensors to monitor SpO₂ levels to aid in detection of one or more disorders such as hypoxemia, deteriorating organ function, wound prone tissues or the like. The insole further includes electrodes that may be utilized to stimulate a foot at or adjacent a wound. An insole according to the present disclosure may be utilized to monitor diabetic wounds, and aid in the healing of diabetic wounds of a foot.

### CROSS REFERENCE TO RELATED APPLICATIONS

This application claims the benefit under 35 USC § 119(e) of U.S. Provisional Patent Application No. 63/423,073, filed Nov. 14, 2022, the contents of which are incorporated herein by reference in their entirety.

### BACKGROUND

Those with diabetes have an increased risk of developing cardiovascular, kidney, eye, and lower-extremity complications. Diabetic people may be prone to developing ulcers on their feet, which can become chronic or heal slowly, potentially resulting in peripheral neuropathy, foot ulceration, and amputation. Sub-optimal oxygenation (hypoxia) may be a factor that interferes with the healing of diabetic wounds by limiting the blood flow rate to the wound.

### SUMMARY

An aspect of the present disclosure is a smart shoe insole that includes photoplethysmography (PPG sensors) to monitor SpO₂ (i.e., blood oxygen saturation) levels. The SpO₂ levels may comprise the ratio of oxygenated hemoglobin to the total hemoglobin in blood. SpO₂ levels may be utilized to aid in detection of one or more disorders such as hypoxemia, deteriorating organ function, wound-prone tissues, etc. An insole according to the present disclosure may be utilized to provide early treatment (e.g., at home). The insole may be configured to monitor diabetic wounds and aid in the healing of diabetic wounds at a foot.

Another aspect of the present disclosure is a method of making an insole for monitoring and treating diabetic foot ulcers. The method includes fabricating a resilient shoe insole and positioning at least one photoplethysmography (PPG sensor) on an upper side of the resilient shoe insole, whereby the PPG sensor can be utilized to monitor arterial oxygen saturation (SpO₂) levels of a patient's foot positioned on the upper side of the resilient shoe insole. The method further includes positioning a plurality of flexible electrodes on the resilient shoe insole in a location that is selected to cause at least one of the plurality of flexible electrodes to be positioned adjacent to a wound on the patient's foot when the patient's foot is positioned on the upper side of the resilient shoe insole. The method further includes operably interconnecting the at least one PPG sensor and the plurality of flexible electrodes to a monitoring unit that is configured to actuate at least one of the plurality of flexible electrodes to stimulate the patient's foot at or adjacent the wound based, at least in part, on information from the at least one PPG sensor.

Another embodiment of the present disclosure is a wearable article for monitoring and healing foot wounds including at least one photoplethysmography (PPG) sensor and at least one electrode to a foot of a patient; whereinat least one photoplethysmography (PPG) sensor of the wearable article to be positioned adjacent to the foot; wherein the at least one electrode of the wearable article is positioned adjacent a wound on the foot; wherein the at least one photoplethysmography (PPG) sensor is capable of providing data for at least (1) determining an SpO2 estimation and (2) calculating a perfusion index; and wherein electrical power is provided to the at least one electrode if: 1) the perfusion index is acceptable according to predefined criteria, and 2) the SpO2 estimation is not acceptable according to predefined criteria. Yet other embodiments include wherein the predefined criteria for the perfusion index comprises a perfusion index above 0.4%; the predefined criteria for the SpO2 comprises an SpO2 level above 92%. Other embodiment further include an electrical heating system capable of providing heat to the patient's foot if the perfusion index is not acceptable according to the predefined criteria. Yet others include an electrical heating system capable of providing heat to the patient's foot if: 1) the perfusion index is acceptable according to predefined criteria, and 2) the SpO2 estimation is not acceptable according to predefined criteria.

In further embodiments, electrical power is provided to the at least one electrode further includes controlling at least one parameter selected from the group consisting of: a pulse duration of a voltage supplied to the at least one electrode, a peak electrical current supplied to the at least one electrode, a waveform of a voltage supplied to the at least one electrode, and a frequency of a voltage supplied to the at least one electrode. Yet other embodiments further include comprising a resilient insole, wherein the at least one PPG sensor is positioned on an upper side of the resilient insole; wherein the at least one electrode comprises a plurality of flexible electrodes positioned on the resilient shoe insole at locations that are selected such that at least one of the plurality of flexible electrodes is positioned adjacent to a wound on the patient's foot when the patient's foot is positioned on the upper side of the resilient shoe insole; and wherein the at least one PPG sensor and the plurality of flexible electrodes are operably connected to a monitoring unit that is configured to actuate at least one of the plurality of flexible electrodes to stimulate the patient's food at or adjacent the wound base, at least in part, on information from the at least one PPG sensor.

These and other features, advantages, and objects of the present device will be further understood and appreciated by those skilled in the art upon studying the following specification, claims, and appended drawings.

### BRIEF DESCRIPTION OF THE DRAWINGS

In the drawings:

FIG. 1 is a partially schematic side-elevational view of a wearable insole positioned inside a boot adjacent a foot;

FIG. 2 is an isometric view of a wearable insole according to an aspect of the present disclosure;

FIG. 3 is a partially schematic exploded view of a wearable insole according to an aspect of the present disclosure;

FIG. 4 is an exploded isometric view showing top and bottom layers of a wearable insole according to an aspect of the present disclosure;

FIG. 5 is a plan view of a bottom layer of a wearable insole according to an aspect of the present disclosure;

FIG. 6 is a plan view of a top layer of a wearable insole according to an aspect of the present disclosure;

FIG. 7 is a top plan view of an electrode assembly according to an aspect of the present disclosure;

FIG. 8 is a cross sectional view of a portion of the electrode assembly of FIG. 7;

FIG. 9 is a top plan view of an overlay according to an aspect of the present disclosure;

FIG. 10 is a partially schematic cross-sectional view of a portion of an insole according to an aspect of the present disclosure;

FIG. 11 is a top plan view of a conductive interconnect according to an aspect of the present disclosure;

FIG. 12 is a top plan view of an overlay according to an aspect of the present disclosure;

FIG. 13 is a top plan view of a bottom layer of a wearable insole according to an aspect of the present disclosure;

FIG. 14 is a cross sectional view of the bottom layer of FIG. 13; and

FIG. 15 is an isometric view showing a wearable insole during assembly according to an aspect of the present disclosure.

FIG. 16 is a flowchart showing a wound monitoring and treatment process.

### DETAILED DESCRIPTION OF EMBODIMENTS

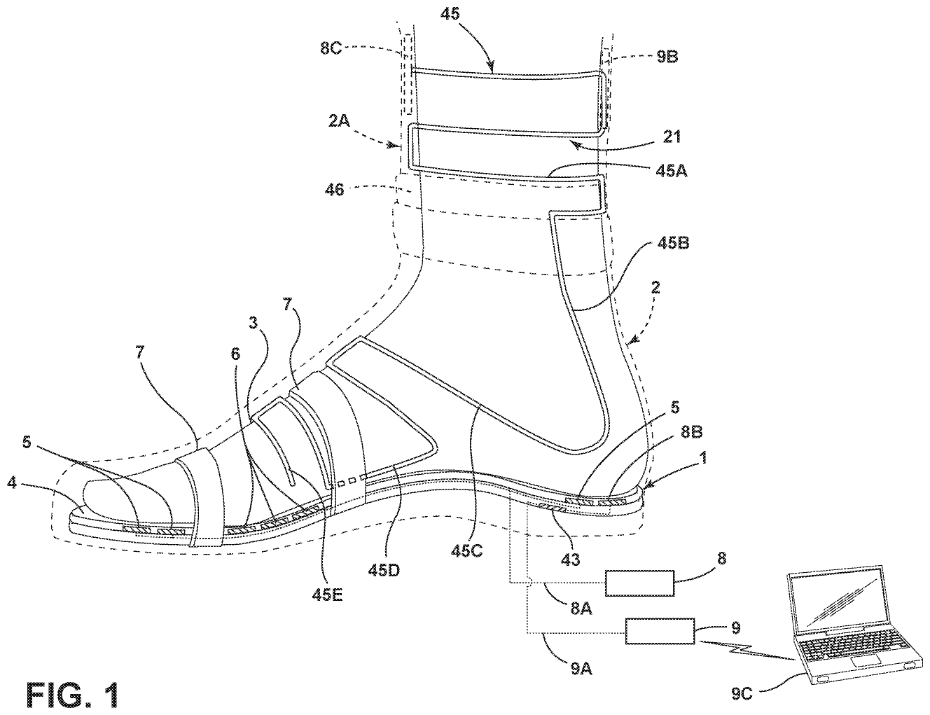

For purposes of description herein the terms “upper,” “lower,” “right,” “left,” “rear,” “front,” “vertical,” “horizontal,” and derivatives thereof shall relate to the device as oriented in FIG. 1. However, it is to be understood that the device may assume various alternative orientations and step sequences, except where expressly specified to the contrary. It is also to be understood that the specific devices and processes illustrated in the attached drawings, and described in the following specification are simply example embodiments of the inventive concepts defined in the appended claims. Hence, specific dimensions and other physical characteristics relating to the embodiments disclosed herein are not to be considered as limiting, unless the claims expressly state otherwise.

With reference to FIG. 1, a wearable insole 1 according to an aspect of the present disclosure may be positioned inside, or attached to, footwear such as boot 2 whereby a foot 3 of a patient contacts an upper side 4 of the insole 1. As discussed in more detail below, the insole 1 may include one or more sensors 5 and one or more electrodes 6. Sensors 5 and electrodes 6 may be configured to contact or engage the foot 3 of a user. The locations and sizes of sensors 5 and/or electrodes 6 may be selected (fabricated) based on the locations of ulcers or other problem areas of a specific patient. The insole 1 may be secured to the foot 3 utilizing straps 7. Straps 7 may comprise hook and loop fasteners to thereby removably secure the wearable insole 1 to a user's foot 3. The sensors 5 and electrodes 6 may be operably connected to a power supply 8 that provides power to the sensors 5 and electrodes 6. Power supply 8 may be electrically interconnected to the sensors 5 and electrodes 6 by conductive lines 8A. Also, the power supply 8 may be positioned inside the boot 2 and/or integrally formed with the insole 1. The sensors 5 and electrodes 6 may also be operably interconnected to a computing device 9. In general, the computing device 9 may be operably interconnected wirelessly, such that a “hard line” 9A is not necessarily required. Also, the computing device 9 may comprise one or more computing devices (e.g., 9C) having processors and the like that are operably interconnected.

The wearable insole 1 may optionally include an electrical heater 21 having a conductive resistance line 45 including segments 45A-45E that extend around a user's foot and/or ankle and/or upper leg. The position and configuration of the conductive line 45 may be adjusted or varied as required for a particular application. The conductive line 45 of electrical heater 21 may be operably connected to power supply 8C, which may be positioned inside an upper portion 2A of the boot 2. The upper portion 2A may comprise a separate component that can be separately attached to form a gap 46 between upper portion 2A and the lower portion of boot 2. Alternatively, the upper portion 2A may be integrally formed with the lower portion of boot 2. An electrical stimulation unit or controller 9B may be positioned in upper portion 2A of boot 2. The electrical stimulation unit 9B may be configured to control the heater 21 and may be configured to communicate with computing device 9. Alternatively, the computing device 9 may be configured to directly control the electrical heater 21. Also, a separate power supply 8C for electrical heater 21 is not necessarily required, and electrical heater 21 may be powered utilizing power supply 8, or power supply 8B.

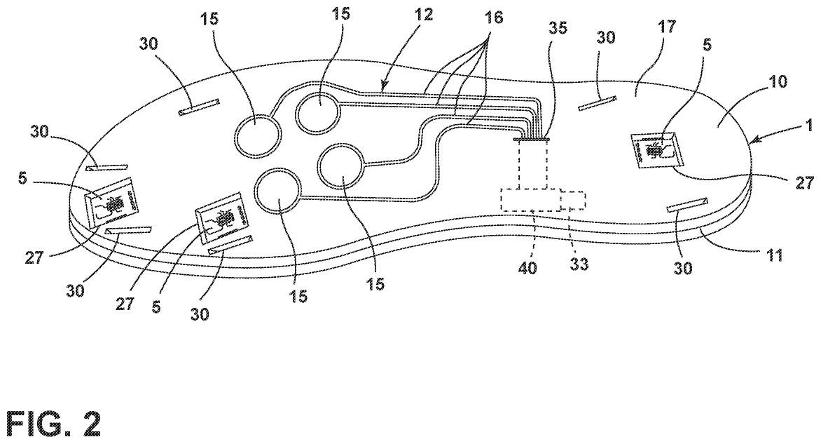

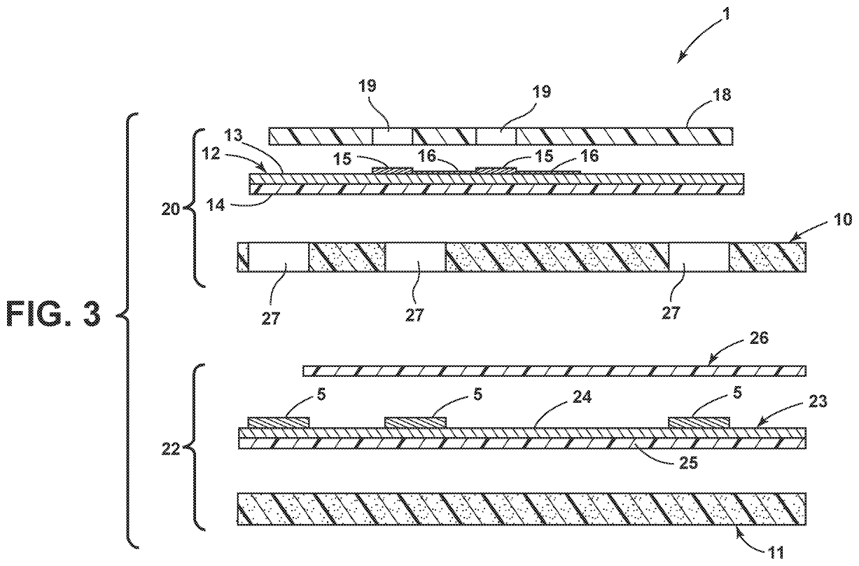

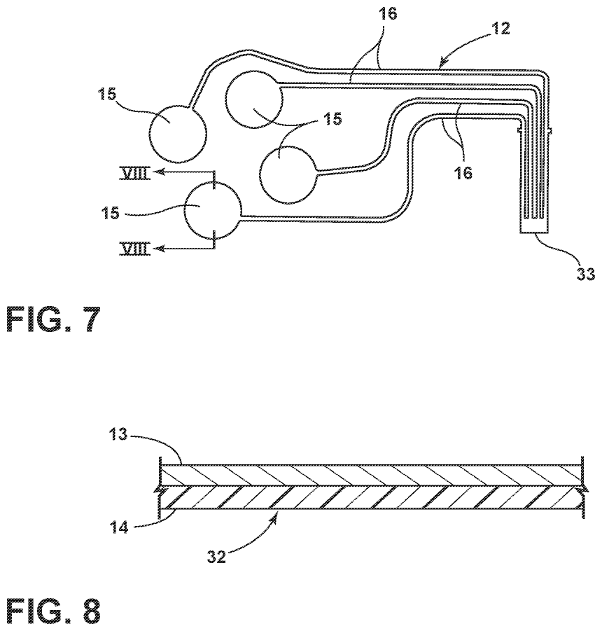

With further reference to FIGS. 2 and 3, wearable insole 1 may include an upper layer 10, and a lower layer 11. The upper and lower layers 10 and 11 may comprise resilient foam or other suitable material to provide comfort for a user. As discussed in more detail below, an electrode 12 (FIG. 3) may include layers 13 and 14 that are bonded together. Layer 13 may be conductive, and lower layer 14 may comprise a polymer such as polyethylene terephthalate (e.g., PET). Electrode 12 may include enlarged areas 15 (FIG. 2) and traces 16. Areas 15 and traces 16 may be disposed on upper side 17 of insole 1 when insole 1 is fully assembled as shown in FIG. 2. The enlarged areas 15 may be circular as shown in FIG. 2, or the enlarged areas 15 may have a different shape as required for a particular application. In general, the electrode 12 and enlarged areas 15 may be customized for each patient for different diabetic foot ulcer (DFU) wound sizes and locations, while also being conformal to the foot 3. The positioning and size of the enlarged areas 15 of electrode 12 may be personalized for each patient based, at least in part, on optical imaging of a TFU wound. The electrode 12 may provide electrical stimulation to the DFU wound to reduce inflammation and/or increase blood flow around the DFU. As discussed in more detail below in connection with FIGS. 7-10, an overlay 18 comprising a polymer such as thermoplastic polyurethane (TPU) may be heat pressed to the upper layer 10 with holes 19 of overlay 18 aligned with the enlarged areas 15 of electrode 12. During fabrication of the insole 1, the upper layer 10, electrode 12, and overlay 18 may be initially fabricated as an upper assembly 20.

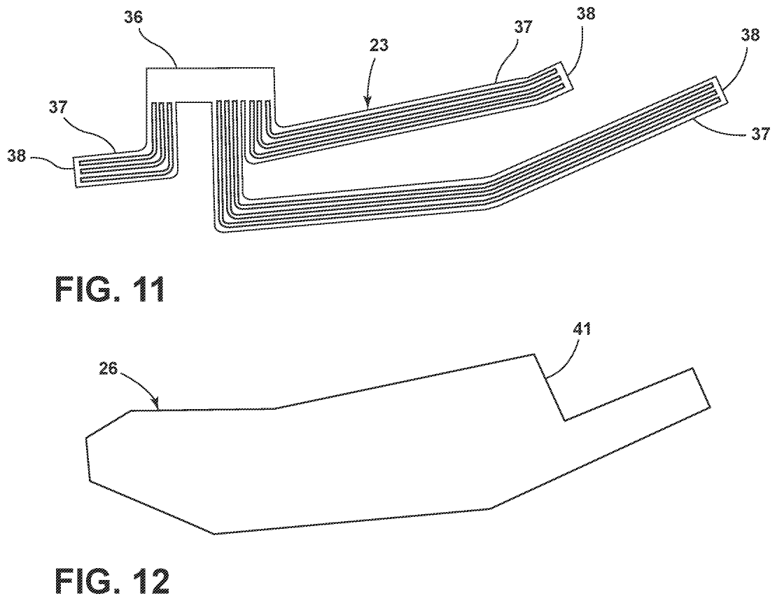

Referring again to FIG. 3, a lower assembly 22 of insole 1 includes photoplethysmography (PPG) sensors 5 which are connected to an interconnect assembly 23. As discussed in more detail below in connection with FIGS. 11-14, interconnect assembly 23 may comprise a conductive upper layer 24 that may be made from a suitable material (e.g. copper), and a lower layer 25 that may comprise a polymer such as TPU. An overlay 26 may be heat pressed to the lower layer 11 to cover at least a portion of the interconnect assembly 23. When assembled, PPG sensors 5 may be positioned in openings 27 of upper layer 10 whereby the PPG sensors 5 may be exposed on upper side 17 of insole 1 as shown in FIG. 2. When assembled, the upper and lower assemblies 20 and 22 may be secured to each other utilizing adhesive that may be applied to the upper and lower layers 10 and 11, respectively.

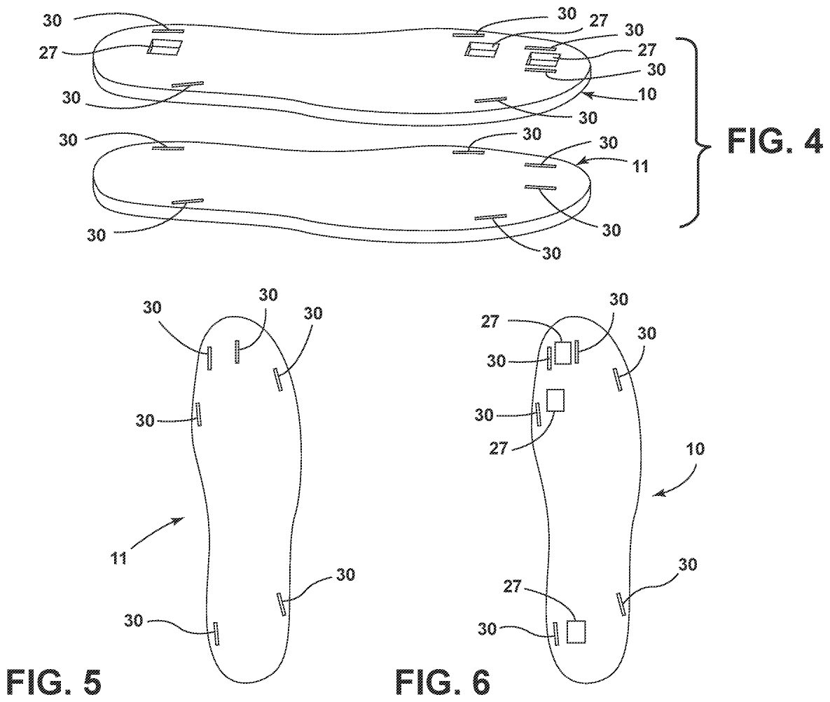

With further reference to FIGS. 4-6, during assembly upper and lower layers 10 and 11 may be cut from layers of foam (e.g. SOF COMFORT double thick foam). A plurality of slots 30 (FIGS. 5 and 6) may be cut in the upper and lower layers 10 and 11. When assembled, straps 7 may extend through the slots 30 to secure the insole 1 to a user's foot. Openings 27 may be cut in upper layer 10 at suitable locations for PPG sensors 5. The upper and lower layers 10 and 11 may be cut utilizing a CO₂ laser or other suitable device and process. The shape and sizes of the upper and lower layers 10 and 11 may be custom fit to correspond to the shape and size of a specific patient's foot. Also, the locations of the openings 27 in upper layer 10 may vary as required for a particular patient to provide for proper positioning of PPG sensors 5.

With further reference to FIGS. 7-10, the electrode 12 may be formed by printing a large area (e.g. square) of silver ink on a PET substrate to form a blank 32 (FIG. 8). The blank includes an upper layer 13 that is conductive, and a lower layer 14 that is non-conductive. The blank 32 may be cured in an oven (e.g. 100° C.) for a period of time (e.g. 10 minutes). In general, the temperature may vary from about 50° C. to about 150° C., and the time may vary as required to cure the ink forming conductive layer 13. In general, the temperature is less than a melting point of the polymer substrate 14.

The blank 32 is then cut to form electrode 12 (FIG. 7). The blank 32 may be cut utilizing a laser or other suitable device and process. As discussed above, electrode 12 generally includes enlarged (e.g. circular) areas 15 and traces 16. Traces 16 may be interconnected at an end 33.

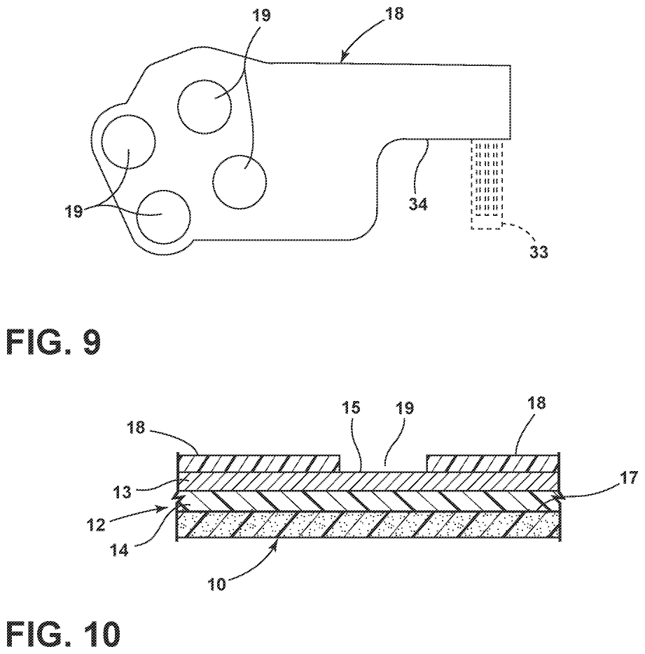

An overlay 18 (FIG. 9) may then be cut from a sheet of polymer material (e.g. TPU). The overlay 18 may include openings 19 that are aligned with the enlarged areas 15 of electrode 12 when insole 1 is assembled such that electrodes 15 are not covered by the overlay 18 when the insole 1 is assembled. Overlay 18 may include an edge 34 having a shape and size that is configured to cover most or all of the traces 16 of electrode 12 without covering the entire upper surface of upper layer 10.

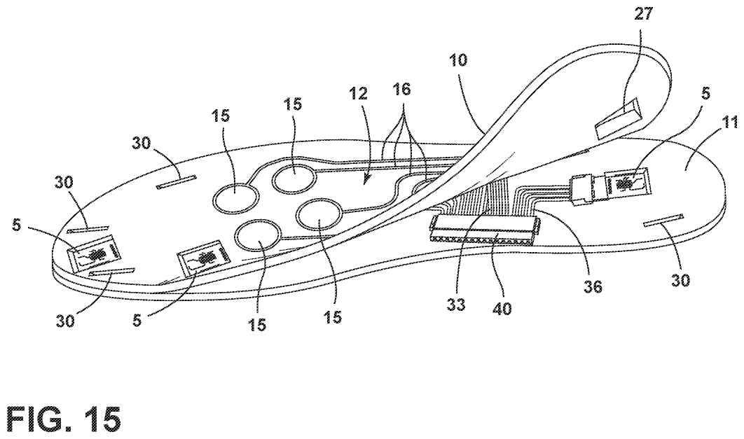

The electrode 12 may then be positioned on upper side 17 of upper layer 10 (FIG. 10), and overlay 18 may then be positioned over the electrode 12. The overlay 18 may then be adhered to the upper layer 10 by heat pressing the overlay 18 onto the upper layer 10. It will be understood that overlay 18 directly contacts upper side 17 of upper layer 10 except in areas where electrode 12 is positioned between overlay 18 and upper layer 10. The end portion 33 of electrode 12 may project beyond edge 34 (FIG. 9) of overlay 18 such that the end 33 can be inserted through a slit 35 (FIG. 2) in upper layer 10 whereby the end 33 can be connected to a pin connector 40 (FIG. 15) that may be positioned between upper and lower layers 10 and 11.

With further reference to FIGS. 11-14, interconnect assembly 23 may be fabricated by adhering conductive material (e.g. copper tape) onto a layer of polymer (e.g. PET), and the interconnect 23 may then be cut utilizing a laser or other suitable device and procedure. In general, the interconnect 23 may include a base 36, and three sets of traces 37, each having an end portion 38 that is configured to be connected to a PPG sensor 5.

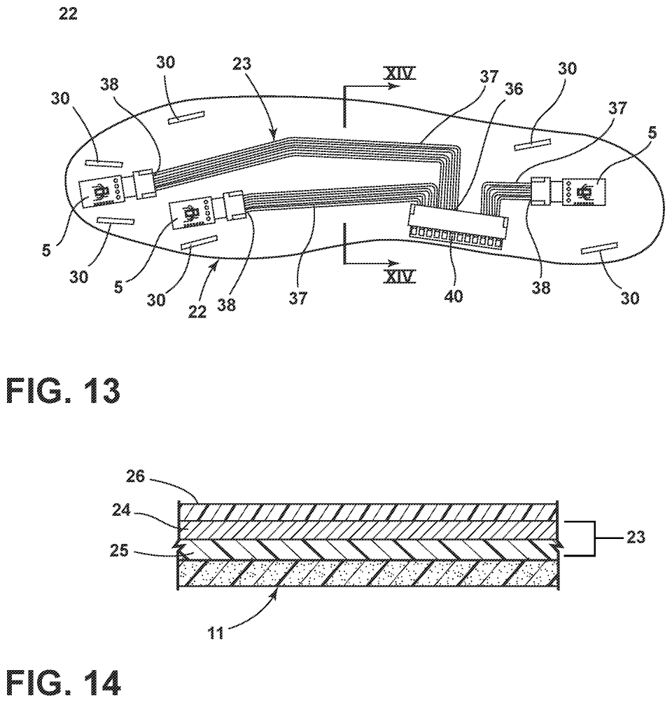

With further to FIG. 12, overlay 26 may be cut from a sheet of polymer (e.g. TPU). The overlay 26 may include an edge 41 having a cut-out portion whereby the base 36 of interconnect assembly 23 is not covered by the overlay 26 when assembled. The interconnect assembly 23 is then positioned on the lower layer 11 (FIGS. 13 and 14), and the overlay 26 is then positioned over the interconnect 23, and the overlay 26 is then heat-pressed to adhere the overlay 26 to the lower layer 11, with the interconnect 23 positioned between lower layer 11 and overlay 26. Pin connector 40 (FIG. 13) may be secured to base 36 of interconnect 23, and PPG sensors 5 may be connected to ends 38 of traces 37.

With further reference to FIG. 15, the end 33 of electrodes 12 may be crimped to pin connector 40. As noted above, the end 33 of electrode 12 may extend through a slit 35 (see also FIG. 2) in upper layer 10 whereby the end 33 of electrode 12 is positioned between upper layer 10 and lower layer 11. The upper and lower layers 10 and 11 may then be secured to each other utilizing adhesive or other suitable material or processes.

Referring again to FIG. 2, when insole 1 is assembled, the enlarged areas 15 of electrode 12 are exposed on an upper side of insole 1, and PPG sensors 5 are also exposed on upper side 17 of insole 1. The number of PPG sensors 5 and the number of electrodes 15 may vary as required for a particular application (e.g. patient). Also, the locations of the PPG sensors 5 and electrodes 15 may be adjusted as required for a particular application or user. In general, the fabrication process noted above is readily adaptable to provide a unique insole 1 as required for a particular patient.

The electrodes 15 may provide electrical stimulation to a wound to reduce inflammation and/or to increase blood flow around a diabetic foot ulcer (DFU) or other wound, and the PPG sensors may be utilized to monitor blood volume changes and the rate of wound healing.

Referring again to FIG. 1, the computing device 9 may comprise a smartphone or other wireless device that may be operably interconnected with the insole 1 utilizing a wireless transmitting and receiving device 43 that is integrated in insole 1 (e.g. inside a boot 2). Similarly, as noted above, power supply 8 may also be positioned inside or on boot 2 as shown at 8A. In general, computing device 9 may be utilized to remotely monitor the progress of a patient. The computing device 9 may be configured to control the electrical stimulation provided by the electrodes according to a pre-programmed schedule. Also, the computing device 9 may be configured to control electrical stimulation of the electrodes based, at least in part, on measurements from PPG sensors 5.

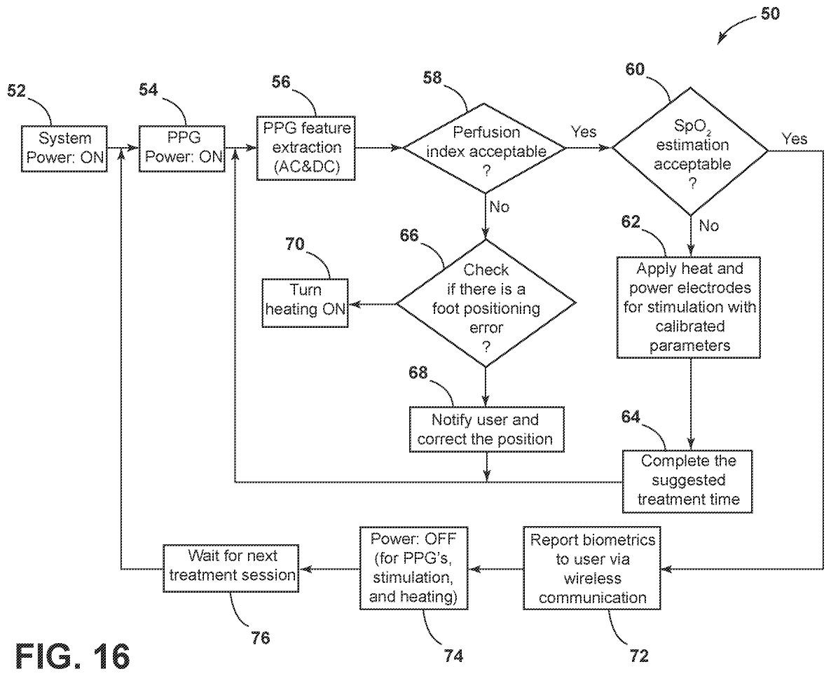

With further reference to FIG. 16, insole 1 may be utilized to continuously monitor (and treat) a wound utilizing a process 50. Process 50 includes measuring the level of the oxygen supply in the tissue bed. As discussed in more detail below, the PPG signal from PPG sensors 15 may be collected (e.g. by computing device 9 or other suitable device), and feature extraction may be performed on the PPG signal. For example, the AC and DC components the PPG signal may be extracted to calculate the blood perfusion index and/or the SpO₂ level. Depending on the oxygen level, the electrodes 6A and/or heating unit 21 may be activated in order to increase the blood flow and thus the oxygen supply to a wound.

Referring again to FIG. 16, at step 52 the system power is turned on to start process 50, and the power to the PPG sensors 5 is turned on at step 54. The AC and DC components of the PPG signal are then extracted at step 56. The perfusion index and SpO2 level are then calculated. At step 58 the perfusion index is evaluated to determine if it is acceptable according to predefined criteria. For example, a perfusion index value of 0.4% may be utilized as an evaluation criteria whereby a perfusion index great than 0.4% is acceptable, and a perfusion index that is 0.4% or below is not acceptable. It will be understood that other values (predefined criteria) for the perfusion index (e.g. 0.3% or 0.5%) may be utilized if appropriate for a particular application, and the present disclosure is not limited to any specific perfusion index criteria.

If the perfusion index is acceptable, the process proceeds from step 58 to step 60 to determine if the SpO₂ estimation is acceptable according to predefined criteria. The predefined criteria may comprise an SpO₂ level above 92%. Other predefined SpO₂ level criteria (e.g. 90% or 94%) may also be utilized, and the present disclosure is not limited to a specific SpO₂ criteria.

If the SpO₂ estimation is acceptable at step 60, the process from continues to step 72. At step 72, the biometrics may be communicated to a user or other entity. The communication may be accomplished utilizing a wireless communication. The power may then be turned off (step 74), and the system may then wait for the next treatment session (step 76).

If, at step 58, the perfusion index is not acceptable, an SpO2 estimation will not be valid. Thus, if the perfusion index is not acceptable at step 58, the process proceeds to step 66, and the system checks to determine if there is a problem such as a foot positioning error. A foot position error may be detected if there is noise in the PPG signal caused by motion artifacts. If the system identifies a foot positioning error at step 66 the user (physician or patient) is notified at step 68, and the foot position is then corrected. The process 50 then returns to step 56. If the system determines that there is no foot positioning error at step 66, the heating unit 21 may be turned on at step 70 to provide controlled heat to increase the blood flow to the foot.

As discussed above, if the perfusion index is acceptable at step 58, the process continues to step 60 to determine if the SpO2 estimation is acceptable. If the SpO₂ estimation is not acceptable at step 60, the process continues to step 62. At step 62 the heating unit 21 may be actuated and/or power may be supplied to the electrodes 6. Preferably, the electrodes 6 are actuated and the heating unit 21 is turned on to provide both electrical stimulation and controlled heating.

The electrodes 6 are integrated into the insole 1 to electrically stimulate the wound region, which in turn improves the blood circulation around the wound. As discussed above, the electrodes 6 may be fabricated by depositing conductive inks or other suitable materials onto a surface of an insole using one or more suitable processes such as additive printing, ablation, etc. The stimulation provided by the electrodes 6 may be user-controlled. For example, computing device 9 may be configured to permit a user to adjust one or more of the intensity (0-30 mA), frequency 1-500 Hz), duty cycle (1-20%), and the wave form (square, sinusoidal, triangular, etc.) of the electric current/power supplied to electrodes 6. It will be understood that the system may be configured to permit these parameters to be individually adjusted. The insole may have a plurality of electrodes, and the coverage of the stimulation provided by the electrodes may be controlled by activating selected electrodes that are farther away, or closer to, the wound region. A custom-built circuit may be integrated into the insole 1 to transform low voltage (.g. 12 volts DC) supplied from a battery (e.g. power supply 8) to high voltages (e.g. up to 150 V), at a high frequency to prevent tissue damage while providing electrical stimulation. The circuit may be configured to switch between active electrodes in preselected patterns (e.g. circular rotation, cross rotation, or user-defined rotation). For example, the electrical stimulation may comprise a pulse duration of 200 microseconds, with a 20 mA peak current, with a symmetrical biphasic square waveform at a frequency of 30 Hz, and an amplitude that is selected to be below an amplitude that could cause muscle contraction. The insole 1 may comprise a boot 2 having an integrated heating unit 21 to further increase the blood flow to the foot 3. The system may include a temperature threshold cut-off feature and a temperature sensor (not shown) inside boot 2 for safety purposes whereby the power supply to the electrical heating unit 21 is turned off if a predefined maximum allowable temperature is reached or exceeded. For example, the controller (computing device 9) may be configured to limit a temperature to a maximum of 100° F. adjacent to a user's foot to avoid burns. The heating unit 21 may comprise either printed elements with resistive or conductive materials, or with light emitting diodes (LEDs).

A detailed description of PPG signal acquisition, SpO₂ estimation, PI measurement, and feature extraction of PPG signals may be found in Panahi et al. (2023) “Development of a Flexible Smart Wearable Oximeter Insole for Monitoring SpO2 Levels of Diabetics' Foot Ulcer” IEEE Journal on Flexible Electronics 2(2) pp 61-70, as well as Panahi et al. (2022) “A Smart Wearable Oximeter Insole for Monitoring SpO2 Levels of Diabetics' Foot Ulcer” 2022 IEEE International Conference on Flexible and Printable Sensors and Systems (FLEPS), Vienna, Austria, 2022, pp. 1-4. These references also include a detailed description of machine learning techniques that may be utilized according to other aspects of the present disclosure. The full text of these references is incorporated herein.

Additionally, unless otherwise specified, it is to be understood that discussion of a particular feature or component extending in or along a given direction or the like does not mean that the feature or component follows a straight line or axis in such a direction or that it only extends in such direction or on such a plane without other directional components or deviations, unless otherwise specified.

It will be understood by one having ordinary skill in the art that construction of the described device and other components is not limited to any specific material. Other exemplary embodiments of the device disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the device as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

What is claimed is:

1. A method of making an insole for monitoring and treating diabetic foot ulcers, the method comprising:
fabricating a resilient shoe insole;
positioning at least one photoplethysmogrphy (PPG) sensor on an upper side of the resilient shoe insole whereby the PPG sensor can be utilized to monitor arterial oxygen saturation (SpO₂) levels of a patient's foot positioned on the upper side of the resilient shoe insole;
positioning a plurality of flexible electrodes on the resilient shoe insole in a location that is selected to cause at least one of the plurality of flexible electrodes to be positioned adjacent to a wound on the patient's foot when the patient's foot is positioned on the upper side of the resilient shoe insole;
operably interconnecting the at least one PPG sensor and the plurality of flexible electrodes to a monitoring unit that is configured to actuate at least one of the plurality of flexible electrodes to stimulate the patient's foot at or adjacent the wound based, at least in part, on information from the at least one PPG sensor.

2. The method of claim 1, wherein:
fabricating the resilient shoe insole includes cutting one or more sheets of resilient material to form an upper insole layer and a lower insole layer; and including:
cutting at least one window through the upper insole layer; and
positioning the PPG sensor in the at least one window.

3. The method of claim 2, including:
positioning the plurality of flexible electrodes between the upper and lower insole layers.

4. The method of claim 3, wherein:
fabricating the plurality of flexible electrodes includes forming a blank by printing of conductive ink onto a substrate comprising a thermoplastic material having a melting temperature; and including:
curing the conductive ink by placing the blank in an oven at a temperature that is less than the melting temperature of the thermoplastic material of the substrate; and including:
followed by utilizing a laser to cut the blank into a one-piece electrode assembly having a base region and a plurality of electrodes, wherein each electrode is interconnected to the base by at least one trace.

5. The method of claim 4, wherein:
at least one of the electrodes has a substantially circular peripheral edge.

6. The method of claim 4, including:
positioning the one-piece electrode assembly onto the upper insole layer;
cutting a sheet of thermoplastic polymer to form an overlay having a plurality of openings therethrough;
positioning the overlay over the one-piece electrode assembly with the plurality of openings of the overlay in alignment with the electrodes, and with a portion of the overlay covering at least a portion of the traces, and wherein the overlay contacts the upper insole layer around the electrodes and around at least a portion of the traces;
bonding the overlay to the upper insole layer using a heat-pressing process whereby at least a portion of the electrodes are not covered by the overlay.

7. A method of treating a wound, the method comprising:
securing a wearable article comprising at least one photoplethysmography (PPG) sensor and at least one electrode to a foot of a patient;
causing at least one photoplethysmography (PPG) sensor of the wearable article to be positioned adjacent to the foot;
causing at least one electrode of the wearable article to be positioned adjacent a wound on the foot;
determine a perfusion index utilizing data from the at least one photoplethysmography (PPG) sensor;
determine an SpO₂ estimation utilizing data from the at least one PPG sensor;
providing electrical power to the at least one electrode if: 1) the perfusion index is acceptable according to predefined criteria, and 2) the SpO₂ estimation is not acceptable according to predefined criteria.

8. The method of claim 7, wherein:
the predefined criteria for the perfusion index comprises a perfusion index above 0.4%.

9. The method of claim 8, wherein:
the predefined criteria for the SpO₂ comprises an SpO₂ level above 92%.

10. The method of claim 7, including:
providing heat to the patient's foot if the perfusion index is not acceptable according to the predefined criteria.

11. The method of claim 7, including:
providing heat to the patient's foot if: 1) the perfusion index is acceptable according to predefined criteria, and 2) the SpO₂ estimation is not acceptable according to predefined criteria.

12. The method of claim 7, wherein:
providing electrical power to the at least one electrode includes controlling at least one parameter selected from the group consisting of: a pulse duration of a voltage supplied to the at least one electrode, a peak electrical current supplied to the at least one electrode, a waveform of a voltage supplied to the at least one electrode, and a frequency of a voltage supplied to the at least one electrode.

13. The method of claim 7, wherein:
the wearable article comprises a resilient insole, and the at least one PPG sensor is positioned on an upper side of the resilient insole;
the at least one electrode comprises a plurality of flexible electrodes positioned on the resilient shoe insole at locations that are selected such that at least one of the plurality of flexible electrodes is positioned adjacent to a wound on the patient's foot when the patient's foot is positioned on the upper side of the resilient shoe insole;
the at least one PPG sensor and the plurality of flexible electrodes are operably connected to a monitoring unit that is configured to actuate at least one of the plurality of flexible electrodes to stimulate the patient's food at or adjacent the wound base, at least in part, on information from the at least one PPG sensor.

14. A wearable article for monitoring and healing foot wounds
comprising at least one photoplethysmography (PPG) sensor and at least one electrode to a foot of a patient; wherein
at least one photoplethysmography (PPG) sensor of the wearable article to be positioned adjacent to the foot;
wherein the at least one electrode of the wearable article is positioned adjacent a wound on the foot;
wherein the at least one photoplethysmography (PPG) sensor is capable of providing data for at least (1) determining an SpO₂ estimation and (2) calculating a perfusion index; and
wherein electrical power is provided to the at least one electrode if: 1) the perfusion index is acceptable according to predefined criteria, and 2) the SpO₂ estimation is not acceptable according to predefined criteria.

15. The wearable article of claim 14, wherein:
the predefined criteria for the perfusion index comprises a perfusion index above 0.4%.

16. The wearable article of claim 15, wherein:
the predefined criteria for the SpO₂ comprises an SpO₂ level above 92%.

17. The wearable article of claim 14, 1 further comprising
an electrical heating system capable of providing heat to the patient's foot if the perfusion index is not acceptable according to the predefined criteria.

18. The wearable article of claim 14 including:
an electrical heating system capable of providing heat to the patient's foot if: 1) the perfusion index is acceptable according to predefined criteria, and 2) the SpO₂ estimation is not acceptable according to predefined criteria.

19. The wearable article of claim 14, wherein:
electrical power is provided to the at least one electrode further includes controlling at least one parameter selected from the group consisting of: a pulse duration of a voltage supplied to the at least one electrode, a peak electrical current supplied to the at least one electrode, a waveform of a voltage supplied to the at least one electrode, and a frequency of a voltage supplied to the at least one electrode.

20. The wearable article of claim 14, further comprising a resilient insole, wherein the at least one PPG sensor is positioned on an upper side of the resilient insole;
wherein the at least one electrode comprises a plurality of flexible electrodes positioned on the resilient shoe insole at locations that are selected such that at least one of the plurality of flexible electrodes is positioned adjacent to a wound on the patient's foot when the patient's foot is positioned on the upper side of the resilient shoe insole; and
wherein the at least one PPG sensor and the plurality of flexible electrodes are operably connected to a monitoring unit that is configured to actuate at least one of the plurality of flexible electrodes to stimulate the patient's food at or adjacent the wound base, at least in part, on information from the at least one PPG sensor.
